(19) United States
(12) Patent Application Publication
(10) Pub. No.: US 2020/0171128 A1
(43) Pub. Date: Jun. 4, 2020

(54) COMPOSITIONS AND METHODS FOR IMPROVING COGNITION

(71) Applicant: The Regents of The University of Colorado, a body Corporate, Denver, CO (US)

(72) Inventors: Huntington Potter, Denver, CO (US); Timothy Boyd, Aurora, CO (US); Ching-Jung Wang, Denver, CO (US); Md. Mahiuddin Ahmed, Denver, CO (US)

(21) Appl. No.: 16/615,044

(22) PCT Filed: May 18, 2018
(86) PCT No.: PCT/US2018/033490

§ 371 Date: Nov. 19, 2019

Related U.S. Application Data
(60) Provisional application No. 62/508,860, filed on May 19, 2017.

(51) Int. Cl. A61K 38/19; A61K 9/00; A61P 25/28

(52) U.S. Cl. CPC A61K 38/193; A61K 9/007; A61K 9/0048; A61K 9/0019; A61K 9/0014; A61P 25/28; A61K 9/0053

(57) ABSTRACT

The present invention provides a method of improving cognition, stabilizing cognition, and/or reversing loss of cognition in a subject in need thereof. In certain embodiments, the subject is administered a therapeutically effective account of GM-CSF or any biologically active derivative or analogue thereof.

### CROSS-REFERENCE TO RELATED APPLICATIONS

The present application claims priority under 35 U.S.C. § 119(e) to U.S. Provisional Application No. 62/508,860, filed May 19, 2017, which-application is incorporated herein by reference in its entirety.

### BACKGROUND OF THE INVENTION

Down syndrome (“DS”), also known as trisomy 21 (and which can also arise as mosaic trisomy 21 or by translocation of part of chromosome 21 to another chromosome), is a human genetic disorder caused by the presence of all or part of a third copy of chromosome 21. DS is typically associated with physical growth delays, characteristic facial features, and mild to moderate intellectual disability.

DS has not been linked to chromosomal abnormalities in the parents of the afflicted individual, who are in general genetically normal (except in the rare cases of translocation Down syndrome, in which a parent carries a balanced/reciprocal translocation involving chromosome 21, leading to some gametes with extra chromosome 21 genetic material). The possibility of a child being born with DS increases with the age of the mother: from <0.1% in 20-year-old mothers to 3% in 45-year-old mothers. There is no known behavioral activity or environmental factor for the parents that is connected with birth of a child afflicted with DS.

There is no cure for DS, and regular screening for health problems common in DS is recommended throughout the afflicted individual's life. DS is often associated with a characteristic profile of cognitive and neurological dysfunctions. Such cognitive dysfunctions include issues relating to thinking and learning, short attention span, poor judgment, impulsive behavior, slow learning, and/or delayed language and speech development, and a tendency to depression, anxiety and autism. These problems hamper development of interpersonal relationships for DS individuals, and reduce their ability to lead independent lives.

There is thus a need in the art for identifying novel compositions and methods for improving cognition, stabilizing cognition, and/or reversing loss of cognition in a DS patient. In certain embodiments, these compositions and methods can be used to improve cognition, stabilize cognition, and/or reverse loss of cognition in a subject that is not afflicted with DS. The present invention addresses and meets this need.

### BRIEF SUMMARY OF THE INVENTION

The present invention provides a method of improving cognition, stabilizing cognition, and/or reversing loss of cognition in a subject afflicted with Down Syndrome (DS). In certain embodiments, the method comprises administering to the subject a therapeutically effective amount of GM-CSF or any biologically active derivative or analogue thereof.

In certain embodiments, the subject has not undergone chemotherapy. In other embodiments, the subject is undergoing chemotherapy. In yet other embodiments, the subject has not developed “chemobrain” and/or does not have “chemobrain.”

In certain embodiments, the subject is not afflicted with Alzheimer's Disease (AD). In other embodiments, the subject is afflicted with AD.

In certain embodiments, the GM-CSF or any biologically active derivative or analogue thereof is the only therapeutically effective compound administered to the subject.

In certain embodiments, the GM-CSF or any biologically active derivative or analogue thereof is the only therapeutically effective compound administered to the subject in a sufficient amount to improve cognition, stabilize cognition, and/or reverse loss of cognition in the subject.

In certain embodiments, the cognition comprises learning, memory, knowledge and learning, attention, working memory, judgment and evaluation, reasoning and computation, problem solving and decision making, comprehension and production of language, recognition memory executive function, ability to learn and execute activities of daily living, and/or ability to recognize and respond appropriately to social clues in other people.

In certain embodiments, the GM-CSF or any biologically active derivative or analogue is active as a hematopoietic growth factor. In other embodiments, the biologically active derivative or analogue is at least one selected from the group consisting of sargramostim, molgramostim, and any methylated, C-amidated, N-acetylated, glycosylated, deglycosylated, partially glycosylated, PEGylated and/or partially PEGylated analogue, or derivative thereof. In yet other embodiments, the biologically active derivative or analogue is sargramostim. In yet other embodiments, the dose of sargramostim administered is about 250 μg/m² per day, 125 μg/m² per day, or 50 μg/m² per day.

In certain embodiments, the administration is performed by at least one route selected from the group consisting of subcutaneous, inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, intramuscular, intradermal, intra-arterial, intravenous, intrabronchial, inhalation, and topical. In other embodiments, the subject is a mammal. In yet other embodiments, the mammal is a human.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1 comprises a set of bar graphs illustrating significant differences in baseline open field test results between Dp16 and wild-type mice. Dp16 mice are a model of Down syndrome because they harbor three copies (instead of the usual two copies) of all of the genes on mouse chromosome 16 that correspond to 102 of the 164 human genes on chromosome 21. Both male and female Dp16 mice (Dp16) showed a significant increase in total distance travelled and velocity of movement relative to wild-type control mice (WT) in the open field test at baseline (8 days before treatment with GM-CSF or saline).

FIG. 2 comprises a bar graph illustrating baseline RAWM experiments, which show that Dp16 male mice exhibit significant learning and memory deficits relative to wild-type control mice. For analysis purposes, Day 2 (testing) data from the RAWM were used for the baseline assessments. After averaging the data from 15 trials using the time values for each individual mouse, the mean times to reach the platform for Dp16 (Dp16) and wild-type control mice (WT) were compared using the Student's t test. A value of P<0.05 was considered statistically significant. In the RAWM test at baseline, Dp16 mice (Dp16) required a significantly longer time to reach the platform than wild-type control mice (WT).

FIG. 3 comprises a set of bar graphs illustrating post-treatment RAWM experiments, which show that treatment with GM-CSF improves memory in both Dp16 male mice and wild-type control mice. To evaluate memory post-treatment, baseline data (Day 2, testing) was compared to the post-treatment data (Day 1, memory test). After averaging the time values for the 15 trials for each individual mouse, the mean time to reach the platform for the different groups of mice at baseline (Day 2 testing) and post-treatment (GM-CSF or saline, Day 1 testing) was compared. Statistical analyses were performed using the Student's t test, and a value of P<0.05 was considered statistically significant. The results of this analysis of post-treatment RAWM tests show that treatment with GM-CSF improves memory in both Dp16 male mice and wild-type control mice.

FIG. 4 comprises a set of bar graphs illustrating post-treatment RAWM experiments, which show that Dp16 and wild-type mice treated with GM-CSF have an improved learning ability. On Day 2 (learning ability test) of the post-treatment RAWM testing, the platform was moved to a new arm (from arm 4 to arm 6), while other maze cues remained unchanged. Then each mouse performed 15 trials to evaluate their learning ability post-treatment. The first 6 trials on Day 2 were considered an early training phase, and were not included in the analysis. The average values of last 9 trials (trials 7-15) were used for analyzing the post-treatment data (Day 2, post-treatment testing), which was then compared to the last 9 trials (trials 7-15) of the baseline data (Day 1, baseline testing) to assess learning ability. Statistical analyses were performed using Student's t-test, and a value of P<0.05 was considered statistically significant.

FIGS. 5A-5C illustrate representative indirect immunofluorescence microscopy images showing the expression patterns for GFAP, a marker for astrocytes, in the hippocampus of wild-type mice treated with saline (WT: Saline), Dp16 mice treated with saline (Dp16: Saline), and Dp16 mice treated with GM-CSF (Dp16: GM-CSF) (n=3 male mice/group). FIGS. 5A-5B: Dp16 mice treated with saline showed an increase in both the numbers and sizes of the clusters of GFAP-stained astrocytes in the hippocampus compared to wild-type mice treated with saline. Astrocytes in the Dp16 mice treated with saline have shorter branches (lack of processing) and denser cell bodies compared to wild-type mice treated with saline. FIGS. 5B-5C: Dp16 mice treated with GM-CSF showed a reduced number of GFAP-positive astrocyte clusters, and a decrease in the size of the GFAP-positive astrocyte clusters compared to Dp16 mice treated with saline. FIGS. 5A & 5C: Although GM-CSF-treated Dp16 mice showed a reduced number of GFAP-positive astrocyte clusters and a decrease in the size of the GFAP-positive astrocyte clusters compared to saline-treated Dp16 mice, the numbers and sizes of the astrocyte clusters in the GM-CSF-treated Dp16 mice still appeared higher than the numbers and sizes of the astrocyte clusters in the saline-treated wild-type mice.

FIGS. 6A-6C illustrate representative indirect immunofluorescence microscopy images, including selected regions where the image is expanded to show zoomed-in views of individual cells (right panels), showing the expression patterns of the astrocyte marker GFAP in the hippocampus of wild-type mice treated with saline (WT: Saline), Dp16 mice treated with saline (Dp16: Saline), and Dp16 mice treated with GM-CSF (Dp16: GM-CSF) (n=3 male mice/group). Dp16 mice treated with saline showed an increase in both the numbers and sizes of abnormal clusters of GFAP-positive astrocytes (FIG. 6B) in the hippocampus compared to wild-type mice treated with saline (FIG. 6A). GFAP-positive astrocytes in the Dp16 mice treated with saline have shorter branches (lack of processing) and denser cell bodies (FIG. 6B) compared to wild-type mice treated with saline (FIG. 6A). Dp16 mice treated with GM-CSF showed a reduced number of the abnormal GFAP-positive astrocyte clusters, and a decrease in the size of the GFAP-stained astrocyte clusters (FIG. 6C) compared to Dp16 mice treated with saline (FIG. 6A). Although GM-CSF-treated Dp16 mice showed a reduced number of GFAP-positive astrocyte clusters and a decrease in the size of the GFAP-positive astrocyte clusters compared to saline-treated Dp16 mice, the numbers and sizes of the astrocyte clusters in the GM-CSF-treated Dp16 mice still appeared higher than the numbers and sizes of the astrocyte clusters in the saline-treated wild-type mice.

FIG. 7 illustrates the results of the quantitation of the clusters of GFAP-positive astrocytes using indirect immunofluorescence microscopy images. In the quantitative studies, the number of GFAP-positive astrocyte clusters in the hippocampus were visually counted, and it was found that Dp16 mice treated with saline (Dp16-Sal) had a significantly higher number of GFAP-positive astrocyte clusters compared to wild-type mice treated with saline (WT-Sal), and that GM-CSF treatment of Dp16 mice (Dp16-GM-CSF) resulted in a significant decrease in the number of GFAP-positive astrocyte clusters compared to the saline-treated Dp16 mice. Although GM-CSF-treated Dp16 mice showed a significant reduction in the number of astrocyte clusters compared to saline-treated Dp16 mice, the number of astrocyte clusters was still significantly higher than in wild-type mice treated with saline. These results suggest that GM-CSF treatment leads to an increase in the formation of astrocyte processes, together with a reduction in the numbers and sizes of astrocyte clusters, in the hippocampus of Dp16 mice treated with GM-CSF compared to Dp16 mice treated with saline. Thus, GM-CSF treatment effectively changes the Dp16 abnormal astrocyte morphology toward the normal wild-type astrocyte morphology.

FIG. 8 shows an immunoblot analysis of choline acetyl transferase (ChAT) in hippocampal lysates (n=5-6 males per group). The hippocampus shows significantly elevated levels of ChAT expression in Dp16 mice treated with saline (Dp16-Sal) compared to wild-type mice treated with saline (WT-Sal). Treatment of Dp16 mice with GM-CSF (Dp16-GM-CSF) did not result in a significant change in the levels of ChAT expression compared to Dp16 mice treated with saline. Similar to Dp16 mice treated with saline, Dp16 mice treated with GM-CSF showed a significantly higher level of ChAT expression than wild-type mice treated with saline. This experiment was repeated twice, and average values were used for the statistical analyses. These results suggest that GM-CSF treatment does not have an effect on cholinergic neuronal changes in the Dp16 mice, and indicate that increased acetylcholine production/signaling is not the explanation for the improved cognition caused by GM-CSF treatment, because Dp16 mice treated with GM-CSF did not show a significant change in ChAT expression compared to Dp16 mice treated with saline.

### DETAILED DESCRIPTION OF THE INVENTION

The invention relates in one aspect to the unexpected discovery that treatment of a subject with GM-CSF (Granulocyte-Macrophage Colony-Simulating Factor), or a biologically active analogue or derivative thereof, improves cognitive function in the subject. In certain embodiments, the subject is afflicted with DS. In other embodiments, the subject is not afflicted with DS. In yet other embodiments, the subject has not been administered chemotherapy, and/or is not being administered chemotherapy.

GM-CSF is a hematopoietic growth factor that stimulates the proliferation and differentiation of hematopoietic progenitor cells. Currently marketed by Genzyme, recombinant human GM-CSF (LEUKINE®/sargramostim) has been FDA approved since 1991 and safely used worldwide for leukopenia. Sargramostim is further used in multiple stem cell transplantation settings, and following induction chemotherapy in patients 55 years and older with acute myelogenous leukemia (AML). The latter treatment shortens time to neutrophil recovery and reduces incidence of severe and life-threatening infections.

Specifically, recombinant human GM-CSF (sargramostim) is a leukocyte growth factor indicated for use in certain situations: following induction chemotherapy in AML, in mobilization and following transplantation of autologous peripheral blood progenitor cells, in myeloid reconstitution after autologous bone marrow transplantation, in myeloid reconstitution after allogeneic bone marrow transplantation, and in bone marrow transplantation failure or engraftment delay. The standard sargramostim treatment is five or seven days a week for three weeks either as a subcutaneous injection or by infusion at a dose of 250 mg/m². Any adverse events (AEs) associated with sargramostim are usually rare mild-to-moderate pyrogenic effects that subside upon reducing the dosage of sargramostim by half or by halting administration. Sargramostim has not been withdrawn from investigation or marketing in any country for any reason related to safety or effectiveness.

The invention should not be construed to be limited to naturally occuring GM-CSF. Rather, any analogue or derivative of GM-CSF that acts as a hematopoietic growth factor is useful within the methods of the invention. Non-limiting examples of analogues or derivatives of GM-CSF comprise sargramostim and/or molgramostim. Further examples comprise any methylated, C-amidated, N-acetylated, glycosylated, deglycosylated and/or partially glycosylated analogue or derivative thereof. Further examples comprise any analogue of GM-CSF that has been modified by addition of polyethylene glycol chains, including GM-CSF derivatives in which the amino acid cysteine has been added in one or more locations or used in place of one or more natural amino acids to change the features of GM-CSF, such as stability in the body or ability to cross the blood brain barrier, or which have been used as the site(s) of addition of poly ethylene glycol (Doherty, et al., 2005, Bioconjug. Chem. 16(5):1291-1298).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder and/or the frequency with which such symptom is experienced by a patient, is reduced.

As used herein, the term “astrocyte” or “astroglia” refers to a star-shaped glial cell in the brain and spinal cord. Astrocytes perform functions such as biochemical support of endothelial cells that form the blood-brain barrier, provision of nutrients to the nervous tissue, maintenance of extracellular ion balance, and a role in the repair and scarring process of the brain and spinal cord following traumatic injuries.

As used herein, the term “astrogliosis” or “astrocytosis” refers to an abnormal increase in the number of astrocytes due to the destruction of nearby neurons from CNS trauma, infection, ischemia, stroke, autoimmune responses, and neurodegenerative disease. Astrogliosis changes the molecular expression and morphology of astrocytes, causing scar formation and, in severe cases, inhibition of axon regeneration.

As used herein, the term “ChAT” or “CAT” refers to choline acetyltransferase, which is a transferase enzyme responsible for the synthesis of the neurotransmitter acetylcholine. ChAT catalyzes the transfer of an acetyl group from the coenzyme acetyl-CoA to choline, yielding acetylcholine (ACh). ChAT is found in high concentration in cholinergic neurons, both in the central nervous system (CNS) and peripheral nervous system (PNS).

In one aspect, the terms “co-administered” and “co-administration” as relating to a subject refer to administering to the subject a compound of the invention or salt thereof along with a compound that may also treat the disorders or diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “cognition” refers to a mental action or process of acquiring knowledge and understanding through thought, experience, and the senses. Processes encompassed by cognition include, in non-limiting examples, knowledge and learning, attention, memory, working memory, judgment and evaluation, reasoning and computation, problem solving and decision making, comprehension and production of language, recognition memory executive function, ability to learn and execute activities of daily living, ability to recognize and respond appropriately to social clues in other people and so forth.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, subcutaneous, intravenous, oral, aerosol, parenteral, ophthalmic, nasal, pulmonary and topical administration.

The term “container” includes any receptacle for holding the pharmaceutical composition or to add protection to manage stability and or water-uptake. For example, in one embodiment, the container is the packaging that contains the pharmaceutical composition such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of a compound or agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “end-feet” refers to enlarged, often club-shaped endings by which axons make synaptic contacts with other nerve cells or with effector cells (muscle or gland cells).

As used herein, the term “GFAP” refers to glial fibrillary acidic protein, which is an intermediate filament protein that is expressed by numerous cell types of the central nervous system (CNS) including astrocytes and ependymal cells. GFAP has also been identified in glomeruli and peritubular fibroblasts taken from certain mammalian cells. GFAP is thought to help to maintain astrocyte mechanical strength, as well as the shape of cells.

As used herein, the term “glymphatic system” (or “glymphatic clearance pathway” or “paravascular system”) refers to a waste clearance pathway for the vertebrate central nervous system (CNS). The pathway comprises a para-arterial influx route for cerebrospinal fluid (CSF) to enter the brain parenchyma, coupled to a clearance mechanism for the removal of interstitial fluid (ISF), and extracellular solutes from the interstitial compartments of the brain and spinal cord. Exchange of solutes between CSF and ISF is driven by arterial pulsation and regulated during sleep by the expansion and contraction of brain extracellular space.

As used herein, the term “GM-CSF” refers to granulocyte-macrophage colony-stimulating factor, also known as colony stimulating factor 2 (CSF2). GM-CSF is a monomeric glycoprotein secreted by macrophages, T cells, mast cells, NK cells, endothelial cells and fibroblasts that functions as a cytokine.

The precursor protein of human GM-CSF has the amino acid sequence of SEQ ID NO:1. The mature protein starts at residue 18 (APA . . . ). In certain embodiments, the mature protein is glycosylated. In other embodiments, the protein is monoglycoslyated, diglycosylated, triglycosylated, tetraglycosylated, pentaglycosylated, or hexaglycosylated.

```
                10 20 30 40
MWLQSLLLLG TVACSISAPA RSPSPSTQPW EHVNAIQEAR
                50 60 70 80
RLLNLSRDTA AEMNETVEVI SEMFDLQEPT CLQTRLELYK
                90 100 110 120
QGLRGSLTKL KGPLTMMASH YKQHCPPTPE TSCATQIITF
              130 140
ESFKENLKDF LLVIPFDCWE PVQE
```

The precursor protein of mouse GM-CSF has the amino acid sequence of SEQ ID NO:2. The mature protein starts at residue 18 (APA . . . ).

```
                10 20 30 40
MWLQNLLFLG IVVYSLSAPT RSPITVTRPW KHVEAIKEAL
                50 60 70 80
NLLDDMPVTL NEEVEVVSNE FSFKKLTCVQ TRLKIFEQGL
                90 100 110 120
RGNFTKLKGA LNMTASYYQT YCPPTPETDC ETQVTTYADF
              130 140
IDSLKTFLTD IPFECKKPGQ K
```

Sargarmostim, marketed by Genzyme under the tradename LEUKINE®, is a recombinant GM-CSF that functions as an immunostimulator. Sargarmostim has the amino acid sequence of SEQ ID NO:3. In certain embodiments, the protein is glycosylated.

```
                10 20 30 40
APARSPSPST QPWEHVNAIQ EALRLLNLSR DTAAEMNETV
                50 60 70 80
EVISEMFDLQ EPTCLQTRLE LYKQGLRGSL TKLKGPLTMM
                90 100 110 120
ASHYKQHCPP TPETSCATQI ITFESFKENL KDFLLVIPFD
              130
CWEPVQE
```

Molgramostim is a recombinant GM-CSF that functions as an immunostimulatory. Molgramostim has the amino acid sequence of SEQ ID NO:4. In certain embodiments, the protein is not glycosylated.

APARSPSPST QPWEHVNAIQ EARRLLNLSR DTAAEMNETV
EVISEMFDLQ EPTCLQTRLE LYKQGLRGSL TKLKGPLTMM
ASHYKQHCPP TPETSCATQI ITFESFKENL KDFLLVIPFD
CWEPVQE

As used herein, the term “hippocampus” refers to the elongated ridges on the floor of each lateral ventricle of the brain, thought to be the center of emotion, memory, and the autonomic nervous system.

As used herein, the term “hypertrophic” refers to enlargement or overgrowth of a cell, tissue, organ, and/or body part due to increased size of the constituent cells.

As used herein, the term “improve” refers to an increase in function, for example, cognitive function, over the level of function exhibited by the animal before administration of the molecules described herein (i.e. GM-CSF and its analogues and derivatives) even if the animal is not exhibiting abnormal function (i.e. is not suffering a disease or condition), but can be considered to be functioning with in the normal range.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “neurogenesis” refers to the process by which nervous system cells, known as neurons, are produced by neural stem cells (NSCs), and it occurs in most species of animals. Types of NSCs include, for example, neuroepithelial cells (NECs), radial glial cells (RGCs), basal progenitors (BPs), intermediate neuronal precursors (INPs), subventricular zone astrocytes, and subgranular zone radial astrocytes, among others. Neurogenesis is most active during embryonic development, and is responsible for producing all the various types of neurons of the organism, but continues throughout adult life in a variety of organisms.

As used herein, the term “neurotrophic” refers to growth of a nervous tissue.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

As used herein, the term “synapse” refers to a structure that permits a neuron (or nerve cell) to pass an electrical or chemical signal to another neuron or to the target efferent cell. Synapses are essential to neuronal function: at a synapse, the plasma membrane of the signal-passing neuron (the presynaptic neuron) comes into close apposition with the membrane of the target (postsynaptic) cell. Both the presynaptic and postsynaptic sites contain elements that link the two membranes together and carry out the signaling process.

As used herein, the term “synaptic integrity” refers to a functional synaptic unit with unimpaired neuronal transmission.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

By the term “specifically bind” or “specifically binds,” as used herein, is meant that a first molecule preferentially binds to a second molecule (e.g., a particular receptor or enzyme), but does not necessarily bind only to that second molecule.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Compounds and Compositions

GM-CSF or any biologically active derivative or analogue thereof is useful within the methods of the invention. In certain embodiments, any analogue or derivative of GM-CSF that acts as a hematopoietic growth factor is useful within the methods of the invention. Non-limiting examples of analogues or derivatives of GM-CSF comprise sargramostim, molgramostim, and any methylated, C-amidated, N-acetylated, glycosylated, deglycosylated and/or partially glycosylated analogue or derivative thereof, or any PEGylated analogue or derivative thereof.

### Salts

The compounds described herein may form salts with acids or bases, and such salts are included in the present invention. In one embodiment, the salts are pharmaceutically acceptable salts. The term “salts” embraces addition salts of free acids or bases that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

### Methods

The present invention provides methods of improving cognition, stabilizing cognition, and/or reversing loss of cognition in a subject, including but not limited to a subject in need thereof. In certain embodiments, the method comprises administering to the subject a therapeutically effective amount of GM-CSF or any biologically active derivative or analogue thereof. In other embodiments, the subject is not afflicted with DS. In yet other embodiments, the subject is not afflicted with Alzheimer's Disease (AD). In yet other embodiments, the subject is afflicted with DS.

In certain embodiments, the subject has not undergone chemotherapy. In other embodiments, the subject is not undergoing chemotherapy. In yet other embodiments, the subject has not developed “chemobrain”, and/or does not have “chemobrain”, due to a prior or concomitant chemotherapy treatment.

In certain embodiments, the subject does not have amyloid deposits in the brain. In yet other embodiments, the subject has amyloid deposits in the brain.

In certain embodiments, the GM-CSF or any biologically active derivative or analogue thereof is the only therapeutically effective compound administered to the subject. In other embodiments, GM-CSF or any biologically active derivative or analogue thereof is the only therapeutically effective compound administered to the subject in a sufficient amount to improve cognition, stabilize cognition, and/or reverse loss of cognition in the subject.

In certain embodiments, the administration is performed by at least one route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical. In other embodiments, the subject is a mammal. In yet other embodiments, the mammal is a human.

The invention further provides a kit comprising GM-CSF or any biologically active derivative or analogue thereof, an applicator and instructional material for use thereof, wherein the instructional material comprises instructions for improving cognition, stabilizing cognition, and/or reversing loss of cognition in a subject, including but not limited to, those in need thereof.

### Combination and Concurrent Therapies

In certain embodiments, the compounds of the invention are useful in the methods of present invention when used concurrently with at least one additional compound useful for preventing and/or treating diseases and/or disorders contemplated herein. In other embodiments, the compounds of the invention are useful in the methods of present invention in combination with at least one additional compound useful for preventing and/or treating diseases and/or disorders contemplated herein. In other embodiments, the compounds of the invention are useful in the methods of the present invention in combination with at least one additional compound useful for improving cognition in an individual not suffering from a disease or disorder, such as but not limited to AD or DS.

These additional compounds may comprise compounds of the present invention or other compounds, such as commercially available compounds, known to treat, prevent, or reduce the symptoms of diseases and/or disorders contemplated herein, and/or improving normal cognition. In certain embodiments, the combination of at least one compound of the invention or a salt thereof, and at least one additional compound useful for preventing and/or treating diseases and/or disorders contemplated herein, or a person not exhibiting a disease or disorder such as those contemplated herein, has additive, complementary or synergistic effects in the prevention and/or treatment of diseases and/or disorders contemplated herein, or improving normal function not reduced or hampered by a disease or disorder such as those contemplated herein.

In another non-limiting example, the compounds of the invention, or a salt or solvate thereof, can be used concurrently or in combination with one or more agents known to be useful in improving and/or prevention loss of cognition, such as cholinesterase inhibitors, neurotransmitter receptor agonists or partial agonists (such as Memantine/Namenda), anti-depressants, and/or anxiolytics.

A synergistic effect may be calculated, for example, using suitable methods such as, for example, the Sigmoid-Eₘₐₓ equation (Holford & Scheiner, 1981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention, or to improve function in an individual not suffering from a disease or disorder such as those contemplated herein. Dosage regimens may be adjusted to provide the optimum and/or beneficial therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic and/or beneficial response for a particular patient, composition, and mode of administration, without being toxic to the patient.

The therapeutically effective amount or dose of a compound of the present invention depends on the age, sex and weight of the patient, the current medical condition of the patient and the progression of a disease or disorder, or the desired level of improved function, contemplated in the invention.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic and/or beneficial effect and gradually increase the dosage until the desired effect is achieved.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, or less than about 0.05 mg, or less than about 0.005 mg, and any and all whole or partial increments thereof.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical administration.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Materials

Dp16 mice, a transgenic model of Down syndrome, carry a partial duplication of mouse chromosome 16, representing the mouse genes that are syntenic to the human genes of chromosome 21 (Gupta, et al., 2016, Mamm Genome 27(11-12): 538-555).

### Example 1: Baseline Behavioral Phenotypes and Behavioral Tasks (Before GM-CSF or Saline Treatment)

Baseline behavior in 12-14-month-old Dp16 mice (11 males and 13 females) and age-matched wild-type control mice (19 males and 13 females) were analyzed. For all behavioral tasks, on the day of testing, mice were brought into the behavior room and allowed to habituate to the room for 30 min. After 30 min, the mice were ready for training and testing. At baseline, the cohorts were subjected to behavioral tasks to assess learning and memory, including: 1) the open field test, which assesses general activity, exploratory behavior, and locomotor activity, at 8 days before the first injection of GM-CSF or saline, and 2) the RAWM (Boyd, et al., 2010, J. Alzheimers Dis. 21(2): 507-518), which assesses working memory, at 6 days before the first injection of GM-CSF or saline.

### Baseline Open Field Test

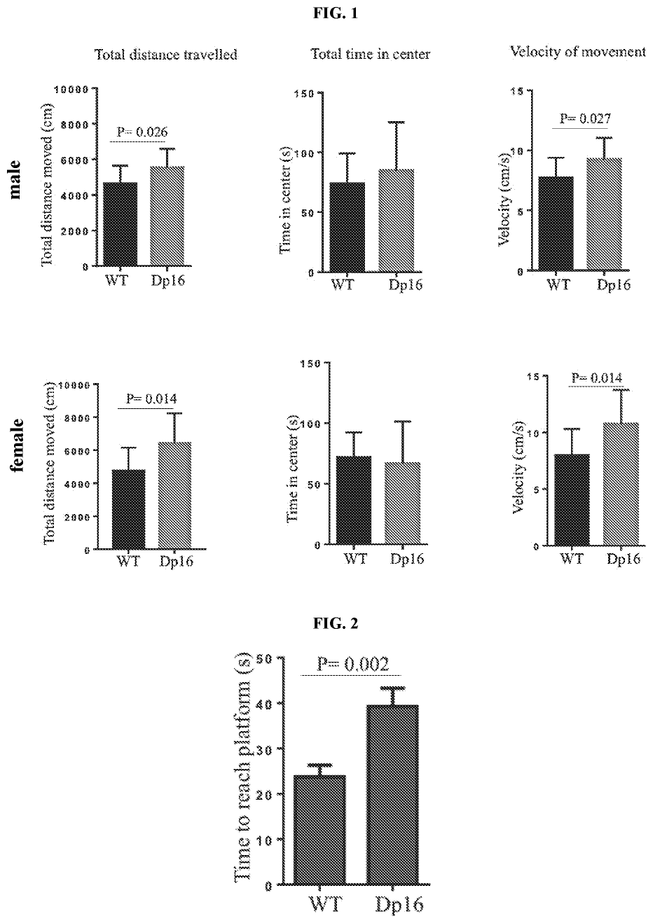

The general activity, exploratory behavior, and locomotor activity in 12-14-month-old Dp16 mice (11 males and 13 females) and their age-matched wild-type control mice (19 males and 13 females) at baseline (8 days before the first injection of GM-CSF or saline) were evaluated by allowing them to freely explore the open field arena for 10 min (slightly modified from Faizi, et al., 2011, Neurobiol Dis 43(2): 397-413). Assessments were performed in a square arena with dimensions of 44 cm×44 cm, and were recorded using a video tracking system. The total distance traveled, total time spent in the center zone, and the velocity of movement were analyzed during the 10 min session. As shown in FIG. 1, both male and female Dp16 mice showed a significant increase in total distance traveled and velocity of movement relative to wild-type control mice in the open field test at baseline (8 days before treatment with GM-CSF or saline).

### Baseline Radial Arm Water Maze (RAWM)

Only male mice were used for the RAWM task (female mice were not tested because of their poor swimming ability). To evaluate the hippocampus-dependent spatial learning and memory task, RAWM (Alamed, et al., 2006, Nat Protoc 1(4):1671-1679) was performed at baseline (6 days before the first injection of GM-CSF or saline). The apparatus for RAWM contains 6 arms and was placed into a circular pool. The maze was placed in a room with numerous cues on the walls and surroundings that the mice could use to navigate the maze. The RAWM utilized a two-day spatial memory task in which a submerged/hidden platform was placed in arm 4 and kept in the same position throughout the testing.

On both Day 1 (learning/training) and Day 2 (testing), 15 trials (60 sec each) were performed for each mouse; they were run as three sessions with a 50 min break between sessions (1^{st} session: 6 trials with 5 min rest between trials; 50 min rest between sessions; 2^{nd} session: 6 trials with 5 min rest between trials; 50 min rest between sessions; and 3^{rd} session: 3 trials with 5 min break between trials). Before beginning the 1^{st} trial, each mouse was allowed to spend 30 sec on the platform to habituate with the walls and surroundings. During each trial, the mouse started from a different arm using a randomization scheme such that during the 15 trials, the mouse started in each of the five arms without the platform three times. On Day 1 (training day), each mouse was gently placed into one of the five arms (not including arm 4, which contained the platform) and was allowed a maximum time of 60 sec to find the platform; if the platform was not found during this time, the mouse was gently guided to it. Once the mouse was on the platform, it was kept there for 10 sec and then quickly towel-dried and transferred to the home cage that was placed on a heating pad for a 5 min break between trials. Between sessions, the mouse rested for 50 min.

On Day 2 (testing day), spatial memory testing was performed in which the platform was placed at the same arm as on Day 1 (arm 4). Similar to Day 1, each mouse was gently placed into one of the 6 arms (but not in arm 4, which contained the platform) and was allowed a maximum time of 60 sec to find the platform. As described, 15 trials (60 sec each) were performed for each mouse; they were run as three sessions with a 50 min break between sessions (1^{st} session: 6 trials with 5 min rest between trials; 50 min rest between sessions; 2^{nd} session: 6 trials with 5 min rest between trials; 50 min rest between sessions; and 3^{rd} session: 3 trials with 5 min break between trials). For analysis purposes, Day 2 (testing) data from the RAWM were used for the baseline assessments. After averaging the data from 15 trials using the time values for each individual mouse, the mean time to reach the platform between Dp16 (Ts) and wild-type control mice (C) was compared using the Student's t test. A value of P<0.05 was considered statistically significant. In the RAWM test at baseline, Dp16 mice (Ts; n=11) required a significantly longer time to reach the platform than wild-type control mice (C; n=17). During each trial, the mouse started from a different randomized arm using the same procedure as on Day 1. Incorrect arm entries were counted as errors for each trial. The mouse rested for 5 min between trials, and for 50 min between sessions. The mouse behavior was recorded with a video camera directly above the water pool.

It is worth noting that these baseline cognitive deficits of the Dp16 mice, as compared to wild-type control mice, are not due to neuronal death caused by the accumulation of any known pathological lesions in the brain, such as Alzheimer's disease amyloid plaques, because the sequence of the mouse amyloid-beta peptide is distinct from that of the human amyloid-beta peptide and further the mouse amyloid-beta peptide does not self-polymerize into fibrils. Therefore, these cognitive deficits in the Dp16 mice must be due to other types of cellular dysfunction, instead of overt pathology-induced neurodegeneration.

### Example 2: Post-Treatment Behavioral Tasks (After GM-CSF or Saline Treatment)

After the baseline pretreatment behavioral testing, the cohorts of Dp16 and wild-type control mice were divided into two treatment groups: the first treatment group of mice was injected subcutaneously with GM-CSF (5 μg/day; 5 days a week, Monday-Friday) for a total of 24 or 25 injections and a total of 32 or 33 days; the second treatment group of mice was injected with saline (200 μl/day; 5 days a week, Monday-Friday) for a total of 24 or 25 injections and a total of 32 or 33 days.

There was a total four groups for both males and females: (1) Dp16 mice injected with saline (3 females; 5 males); (2) Dp16 mice injected with GM-CSF (3 females; 6 males), (3) wild-type mice injected with saline (3 females; 9 males), and (4) wild-type mice injected with GM-CSF (4 females; 8 males). The behavioral phenotypes of male mice (injection days 21, 22, and 23) were evaluated post-treatment using the RAWM (female Dp16 mice were unable to swim and could not be tested) as described for the baseline measurements, with a slight alteration on Day 2 in that the location of the platform was changed from arm 4 (where it was located on Day 1) to arm 6 in order to test the ability of the mice to learn a new task.

### Post-Treatment RAWM

For post-treatment RAWM testing of male mice, to evaluate the memory task after GM-CSF or saline injection, on Day 1 (memory test) of post-treatment testing, the same protocol as at baseline testing (before injection of GM-CSF or saline) was used, including 15 trials for each mouse with the platform placed at arm 4 (the same position used in the baseline RAWM testing). Similar to baseline, on Day 1 of the post-treatment testing, 15 trials (60 sec each) were performed for each mouse; they were run as three sessions with a 50 min break between sessions (1^{st} session: 6 trials with 5 min rest between trials; 50 min rest between sessions; 2^{nd} session: 6 trials with 5 min rest between trials; 50 min rest between sessions; and 3^{rd} session: 3 trials with 5 min break between trials). Before beginning the 1^{st} trial, each mouse was allowed to spend 30 sec on the platform to habituate with the walls and surroundings. During each trial, the mouse was started from a different arm using the same randomization approach described for the baseline experiments.

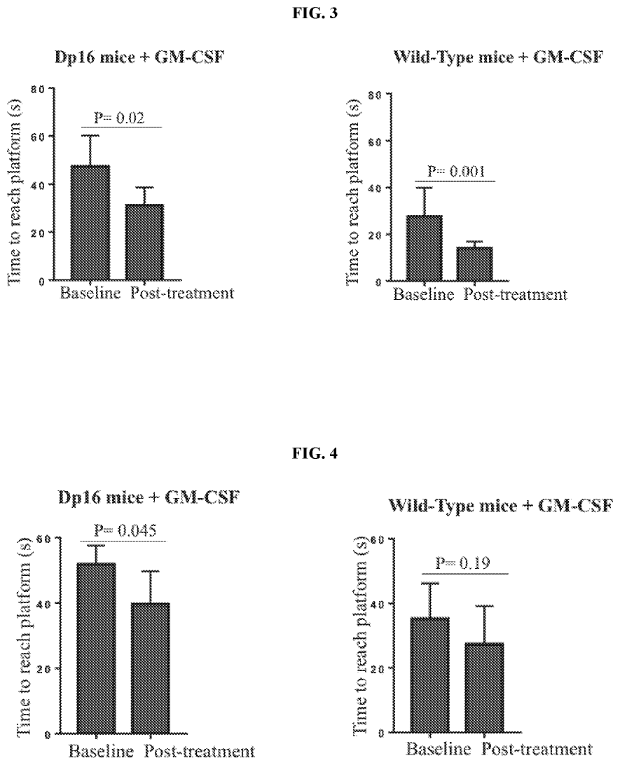

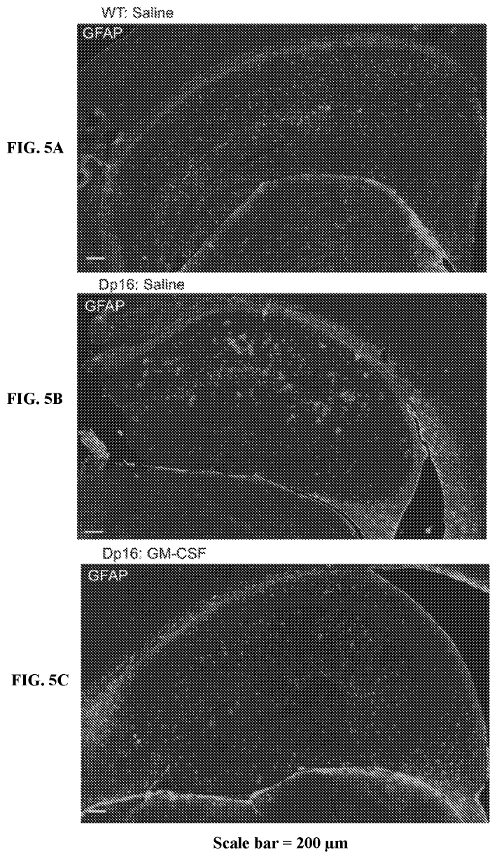

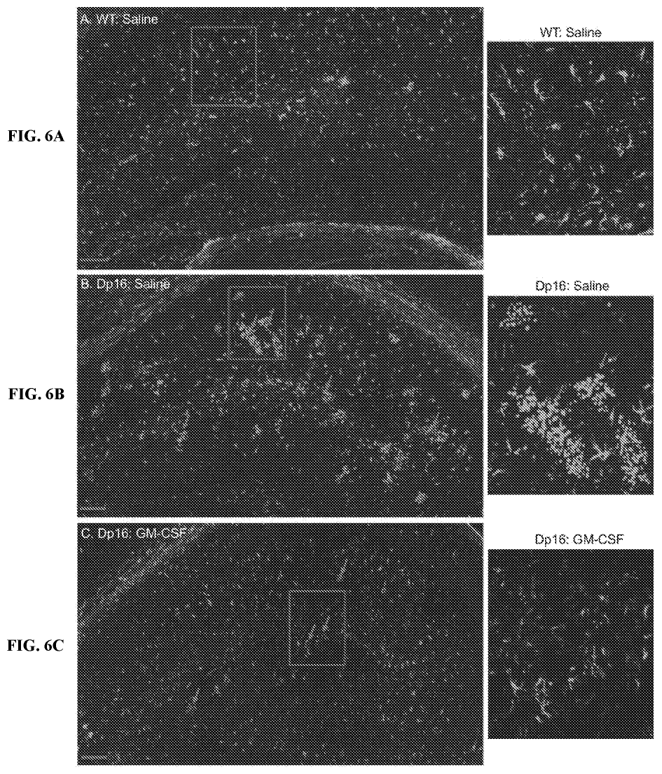

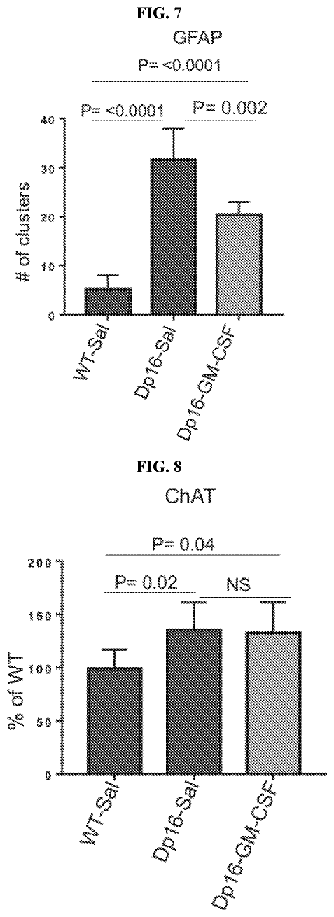

To evaluate memory post-treatment, baseline data (Day 2, testing) was compared to the post-treatment data (Day 1, memory test). After averaging the time values for the 15 trials for each individual mouse, the mean time to reach the platform for the different groups of mice before treatment (baseline, Day 2 testing) and post-treatment (post-treatment, GM-CSF or saline, Day 1 testing) was compared. Statistical analyses were performed using the Student's t test, and a value of P<0.05 was considered statistically significant. As shown in FIG. 3, the results of this analysis of post-treatment RAWM tests show that treatment with GM-CSF improves memory in both Dp16 male mice and wild-type control mice.

On Day 2 (learning ability test) of the post-treatment testing, the platform was moved to a new arm (from arm 4 to arm 6) while other maze cues remained unchanged, and then each mouse performed 15 trials to evaluate their learning ability post-treatment. The first 6 trials on Day 2 were considered an early training phase, and were not included in the analysis. The average values of last 9 trials (trials 7-15) were used for analyzing the post-treatment data (Day 2, post-treatment testing), which was then compared to the last 9 trials (trials 7-15) of the baseline data (Day 1, baseline testing) to assess learning ability. Statistical analyses were performed using Student's t-test, and a value of P<0.05 was considered statistically significant. As shown in FIG. 4, both Dp16 and wild-type mice treated with GM-CSF show an improved learning ability.

### Example 3: Post-Treatment Histological and Immunoblot Analyses (After GM-CSF or Saline Treatment)

As described in Example 2, after the baseline pretreatment behavioral testing, the Dp16 and wild-type control mice were divided into two treatment groups: the first treatment group of mice was injected subcutaneously with GM-CSF (5 μg/day; 5 days a week, Monday-Friday) for a total of 24 or 25 injections and a total of 32 or 33 days; the second treatment group of mice was injected with saline (200 μl/day; 5 days a week, Monday-Friday) for a total of 24 or 25 injections and a total of 32 or 33 days. There was a total of three groups used for histological and immunoblot analyses: (1) Dp16 mice injected with saline; (2) Dp16 mice injected with GM-CSF; and (3) wild-type mice injected with saline.

Following the post-treatment behavior assessments (described in Example 2), mice were anesthetized with sodium pentobarbital, perfused intracardially with saline for 5 min, and the brains were then removed rapidly. The left hemisphere was dissected, frozen immediately in liquid nitrogen, and then stored at −80° C. until it was used for lysate preparation. The right hemisphere was immersed in freshly prepared 4% paraformaldehyde (PFA) in PBS for 24 hours at 4° C. After fixing with PFA, 4 μm paraffin-embedded hippocampal brain sections were used for indirect immunofluorescence microscopy staining.

### Ki67 and Nestin Staining in Hippocampus

It was investigated whether GM-CSF has a direct effect on neurogenesis in the brain. Hippocampal brain sections from wild-type mice injected with saline, Dp16 mice injected with saline, and Dp16 mice injected with GM-CSF were concurrently stained with anti-Ki67 and anti-Nestin antibodies (Abcam, MA, USA). The presence of neurogenesis can be indicated when cells show positive staining for both antibodies that co-localizes. This experiment did not provide evidence of neurogenesis. Without wishing to be limited by any theory, it may not be physiologically likely for an induction of neurogenesis and the generation of functional new neurons to occur, because any new neurons that might arise would also need to migrate and grow their axons correctly in order to integrate into the existing neuronal network, thereby providing improved cognitive function in only around 33 days.

### ChAT Levels in Hippocampus

Another positive explanation for the cognitive improvements observed in the GM-CSF-treated mice can come from strengthened signaling between neurons by increased acetylcholine (ACh) neurotransmitter release into the synapses. Therefore, it was examined whether the level of choline acetyltransferase (ChAT), the enzyme that synthesizes ACh, was increased. GM-CSF can induce ChAT activity in cell culture. However, it is also known that increased consumption of choline, choline transport across the blood brain barrier, and then neuronal uptake of choline are necessary to prevent toxic cell membrane depletion of phospholipids caused by increased ChAT activity. Surprisingly, it was found that ChAT expression levels, and likely ChAT activity levels, were actually increased significantly in the Dp16 mice treated with saline, as compared to wild-type control mice treated with saline. Without wishing to be limited by any theory, if this phenomenon is widespread and chronically overactive in the brains of people with DS, it can potentially be an underlying cellular mechanism predisposing them to increased neuronal apoptosis and cognitive impairment. Also, Dp16 mice treated with GM-CSF did not show a significant change in ChAT expression levels compared to Dp16 mice treated with saline, indicating that increased acetylcholine production/signaling is not the explanation for the improved cognition caused by GM-CSF treatment.

For whole lysate preparation, hippocampal tissues were removed from the −80° C. storage, weighed, and placed in 10 volumes of Iso Electric Focusing (IEF) buffer [8 M Urea, 4% 3-[(3 cholamidopropyl dimethylammonio]-1-propanesulfonate (CHAPS), 50 mM Tris] and homogenized by sonication (3 bursts in a 5 second duration) followed by centrifugation at 14,000 rpm for 30 minutes to remove debris. The supernatant was collected and protein concentrations were determined using the Pierce 660 nm Protein Assay kit (Thermo Scientific, IL, USA). Twenty micrograms of protein lysates per lane were separated using sodium dodecyl sulfate polyacrylamide gel electrophoresis (SD S-PAGE). Following electrophoresis, proteins were transferred onto a polyvinylidene difluoride (PVDF) membrane and were blocked with 5% skim milk in TBST (Tris-buffered saline, 0.1% Tween-20), followed by overnight incubation at 4° C. with anti-choline acetyltransferase (ChAT) antibody (Abcam, MA, USA). Detection of bound primary antibodies was performed using alkaline phosphatase-conjugated goat anti-rabbit (Cell signaling Technology, MA, USA) or alkaline phosphatase-conjugated goat anti-mouse (Cell Signaling Technology, MA, USA) secondary antibodies. Chemiluminescent signals were detected using CDP-Star Chemiluminescence reagent (PerkinElmer, MA, USA); imaging and band intensity quantitation were carried out using the ChemiDoc Imaging Systems and Image lab software (Bio-Rad, CA, USA). All membranes were stripped in 0.2 M glycine pH 2.5/0.05% Tween 20, at 70° C. for 20 min, and re-probed with an anti-actin antibody (Sigma, Saint Louis, Mo., USA) for normalization. For comparisons, data were analyzed using GraphPad Prism 7 software (San Diego, Calif., USA). The unpaired Student t-test was used for statistical evaluation, and a P-value of 0.05 was considered statistically significant.

### GFAP Staining in Hippocampus

Considering that no evidence of GM-CSF-induced neurogenesis was observed and that the neurons did not increase signaling through induction of ACh neurotransmitter production, another hypothesis is that the improved cognitive functioning can be due to enhanced synaptic architecture, in which ACh can remain in the synapse longer by not readily diffusing out of it. Astrocytes are a type of glial cell whose processes are known to extend and encapsulate neuronal synapses, and, thus, increasing their numbers and/or their branching or extension of processes could likely improve synapse architecture so as to effect improved Ach signaling. Astrocytes are also known to provide a myriad of critical roles to aid in proper neuronal function and cognition, such as their uptake of synaptic glutamate to prevent excitotoxicity through the NMDA receptor; or their subsequent release of lactate back to neurons for energy and oxidative metabolism (Stobart & Anderson, 2013, Front Cell. Neurosci. 7:38), or expression of the aquaporin-4 channel in their vascular end-feet for solute flow and removal of toxic metabolites through the glymphatic system (Plog & Nedergaard, 2018, Annu. Rev. Pathol. 13:379-394). With numerous other pro-cognitive effects described for astrocytes, it was investigates whether GM-CSF had a direct effect on astrocyte numbers and morphology (e.g., branching, process extension, and so forth).

Using an antibody against the glial fibrillary acidic protein (GFAP), Dp16 mice treated with saline, as compared to wild-type mice treated with saline, had staining indicative of severe diffuse reactive astrogliosis, with numerous large clusters of astrocytes that extended relatively few processes. GM-CSF treatment of Dp16 mice was found to reversed this abnormal astrocytic morphology such that the astrocytes became less hypertrophic GM-CSF treatment of Dp16 mice was found to increase the arborization of the astrocyte cell processes, and GM-CSF treatment of Dp16 mice resulted in a reduction in the sizes of and in the numbers of the clusters of hypertrophic astrocytes. These direct effects of GM-CSF on reducing the reactive astrogliosis in Dp16 mice could thereby allow the astrocytes to function better in enhancing and maintaining synaptic integrity, and in providing other various neurotrophic functions.

Hippocampal sections were incubated with anti-GFAP antibody (Abcam, MA, USA), followed by incubation with Alexa-Fluor 488-conjugated anti-rabbit secondary antibody (Invitrogen, CA, USA). Slides were mounted using 4′,6-diamidino-2-phenylindole (DAPI) Fluoromount-G (SouthernBiotech) and sealed with a coverslip. The preparation and staining of sections from each mouse brain was repeated 2-4 times with similar results. All sections were imaged using an Olympus IX83 Fluorescence microscope. All images were taken at 20× magnification using the same exposure time.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of improving cognition, stabilizing cognition, or reversing loss of cognition in a subject afflicted with Down Syndrome (DS), the method comprising administering to the subject a therapeutically effective amount of GM-CSF or any biologically active derivative or analogue thereof.

2. The method of claim 1, wherein the subject has not undergone chemotherapy.

3. The method of claim 1, wherein the subject is undergoing chemotherapy.

4. The method of claim 3, wherein the subject has not developed “chemobrain” or does not have “chemobrain.”

5. The method of claim 1, wherein the subject is not afflicted with Alzheimer's Disease (AD).

6. The method of claim 1, wherein the subject is afflicted with AD.

7. The method of claim 1, wherein the GM-CSF or any biologically active derivative or analogue thereof is the only therapeutically effective compound administered to the subject.

8. The method of claim 1, wherein the GM-CSF or any biologically active derivative or analogue thereof is the only therapeutically effective compound administered to the subject in a sufficient amount to improve cognition, stabilize cognition, or reverse loss of cognition in the subject.

9. The method of claim 1, wherein the cognition comprises learning, memory, knowledge and learning, attention, working memory, judgment and evaluation, reasoning and computation, problem solving and decision making, comprehension and production of language, recognition memory executive function, ability to learn and execute activities of daily living, or ability to recognize and respond appropriately to social clues in other people.

10. The method of claim 1, wherein the GM-CSF or any biologically active derivative or analogue is active as a hematopoietic growth factor.

11. The method of claim 1, wherein the biologically active derivative or analogue is at least one selected from the group consisting of sargramostim, molgramostim, and any methylated, C-amidated, N-acetylated, glycosylated, deglycosylated, partially glycosylated, PEGylated or partially PEGylated analogue, or derivative thereof.

12. The method of claim 1, wherein the biologically active derivative or analogue is sargramostim.

13. The method of claim 12, wherein the dose of sargramostim administered is about 250 μg/m² per day, 125 μg/m² per day, or 50 μg/m² per day.

14. The method of claim 1, wherein the administration is performed by at least one route selected from the group consisting of subcutaneous, inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, intramuscular, intradermal, intra-arterial, intravenous, intrabronchial, inhalation, and topical.

15. The method of claim 1, wherein the subject is a mammal.

16. The method of claim 15, wherein the mammal is a human.


SEQUENCE LISTING (4 sequences)

SEQ ID NO: 1 - protein, 144 aa, Artificial Sequence
  Chemically synthesized;  precursor protein of human GM-CSF
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 2 - protein, 141 aa, Artificial Sequence
  Chemically synthesized;  precursor protein of mouse GM-CSF
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 3 - protein, 127 aa, Artificial Sequence
  Chemically synthesized;  sargarmostim
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 4 - protein, 127 aa, Artificial Sequence [sequence omitted]